(19) United States
(12) Patent Application Publication
(10) Pub. No.: US 2025/0076276 A1
(43) Pub. Date: Mar. 6, 2025

(54) Concrete Specimen, Manufacturing Method of Concrete Specimen and Hydrogen Amount Measurement Method

(71) Applicant: Nippon Telegraph and Telephone Corporation, Tokyo (JP)

(72) Inventors: Ryuta Ishii, Musashino-shi, Tokyo (JP); Takuya Kamisho, Musashino-shi, Tokyo (JP); Akira Sugiyama, Musashino-shi, Tokyo (JP)

(21) Appl. No.: 18/727,312

(22) PCT Filed: Jan. 13, 2022
(86) PCT No.: PCT/JP2022/000868

§ 371 Date: Jul. 8, 2024

(51) Int. Cl. G01N 33/38; G01N 1/28; G01N 17/02; G01N 27/26

(52) U.S. Cl. CPC G01N 33/383; G01N 1/28; G01N 17/02; G01N 27/26

(57) ABSTRACT

A concrete test piece includes concrete having a planar first surface, a metal embedded in the first surface, and a nickel film or a palladium film formed on the first surface including the metal.

### TECHNICAL FIELD

The present invention relates to a concrete test piece, a production method for the concrete test piece, and a method for measuring a hydrogen content.

### BACKGROUND ART

Reinforcing bars are embedded in reinforced concrete structures such as infrastructure facilities. Since the reinforced concrete structure is disposed outdoors, hydrogen generated by a corrosion reaction penetrates a metal used for the reinforcing bar. When an amount of the hydrogen penetrating the metal increases, the probability of deterioration and fracture of the metal increases. Therefore, a noninvasive inspection of the deterioration of the reinforcing bar by measuring the amount of hydrogen penetrating the metal is performed.

For example, a test device disclosed in Non Patent Literature 1 is known as a method for measuring the amount of hydrogen penetrating the metal.

According to the test device disclosed in Non Patent Literature 1, one surface of a metal plate to be tested is placed in a corrosive environment as a hydrogen penetrating surface. The other surface of the metal plate is placed in a sodium hydroxide solution as a hydrogen detection surface. When hydrogen penetrating the metal plate through the hydrogen penetrating surface diffuses inside the metal plate and reaches the hydrogen detection surface, the hydrogen is electrochemically and forcibly ionized with the sodium hydroxide solution. The test device can detect a current caused by electrons emitted when the hydrogen is ionized, and can calculate the amount of the hydrogen penetrating the metal plate based on the detected current.

### CITATION LIST

### Non Patent Literature

Non Patent Literature 1: Sakai et al., “An adaptive interval adjustment method for an agent-based monitoring system”, Institute of Electronics, Information and Communication Engineers (IEICE) Technical Report, vol. 119, no. 299, ICM2019-22, pp. 7-12, November 2019.

### SUMMARY OF INVENTION

### Technical Problem

Although the test device disclosed in Non Patent Literature 1 uses a metal foil or the metal plate as a test object of a hydrogen permeation test, there is no mention of using concrete in which the metal plate is embedded (hereinafter, referred to as a “concrete test piece”) as the test object.

The metal plate functions as a partition plate for preventing leakage of the sodium hydroxide solution in contact with the hydrogen detection surface in addition to functioning as the test object. Since the sodium hydroxide solution permeates the concrete, the sodium hydroxide solution leaks even if the concrete test piece can be disposed in the test device. For this reason, there is a problem that the hydrogen permeation test using the concrete test piece cannot be performed.

The present invention has been made in view of the above circumstances, and an object of the present invention is to provide a concrete test piece capable of preventing a permeation of a sodium hydroxide solution, a method for producing the concrete test piece, and a method for measuring a hydrogen content capable of measuring the amount of hydrogen penetrating a metal in the concrete test piece.

### Solution to Problem

A concrete test piece according to an aspect of the present invention includes: a concrete having a planar first surface; a metal embedded in the first surface; and a metal film formed on the first surface including the metal.

A method for producing a concrete test piece according to an aspect of the invention includes the steps of: cutting a concrete block in which a metal is embedded in a concrete on a plane including the metal to provide a concrete piece; and forming a metal film on the metal and the concrete in a cut surface on the concrete piece.

A method for measuring a hydrogen content according to an aspect of the present invention measures an amount of hydrogen penetrating the metal in the concrete test piece described above, and the method includes the steps of: immersing the first surface of the concrete in a sodium hydroxide solution and placing a second surface opposite the first surface in an outdoor environment to detect current flowing in the sodium hydroxide solution; and calculating the amount of the hydrogen penetrating the metal based on the detected current.

### Advantageous Effects of Invention

According to the present invention, it is possible to measure the amount of the hydrogen penetrating the metal in the concrete test piece while preventing the permeation of the sodium hydroxide solution.

### BRIEF DESCRIPTION OF DRAWINGS

FIG. 1A is a cross-sectional view of a concrete block in which a metal is embedded.

FIG. 1B is an explanatory drawing illustrating a cut surface L1 when a concrete block is cut to provide a concrete test piece.

FIG. 1C is a cross-sectional view of a concrete piece cut along the cut surface L1.

FIG. 2A is an explanatory drawing schematically showing a device for forming a metal film on a cut surface of a concrete piece using a vacuum evaporation method.

FIG. 2B is an explanatory drawing illustrating a concrete test piece provided by forming the metal film.

FIG. 3 is an explanatory drawing schematically showing a configuration of a hydrogen content measurement device.

FIG. 4 is an explanatory drawing illustrating a state in which a sodium hydroxide solution is in contact with a first surface of a concrete test piece.

### DESCRIPTION OF EMBODIMENTS

Hereinafter, an embodiment of the present invention will be described with reference to the drawings. A concrete test piece according to the present embodiment is produced by cutting a concrete block in which a metal is embedded on a plane including the metal to provide a concrete piece, and forming a metal film of nickel, palladium, or the like on a cut surface of the concrete piece. The concrete test piece is disposed in a test device such that a surface of the concrete test piece on which the metal film is formed is specified as a hydrogen detection surface (first surface to be described later) and a surface opposite to the hydrogen detection surface is specified as a hydrogen penetrating surface (second surface to be described later), and the amount of the hydrogen that penetrates the metal is measured. Details will be described below.

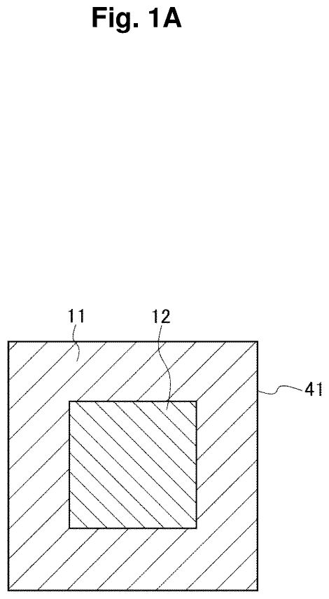

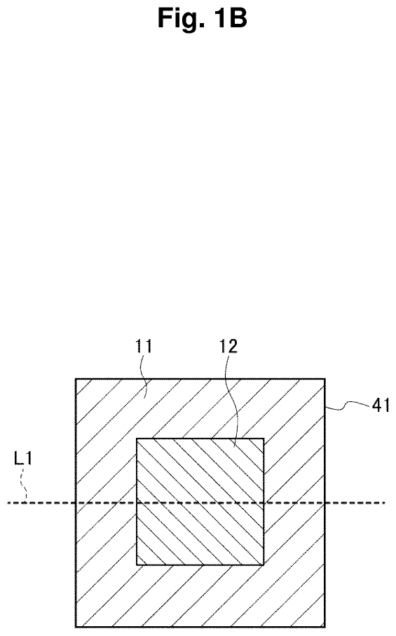

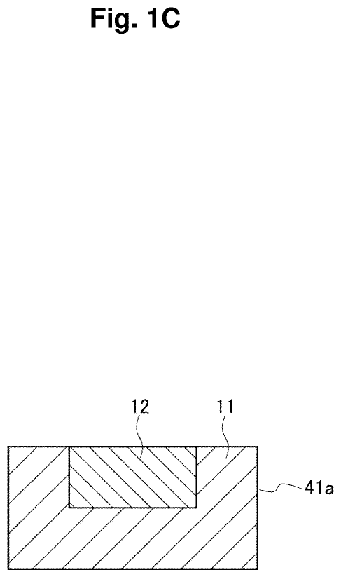

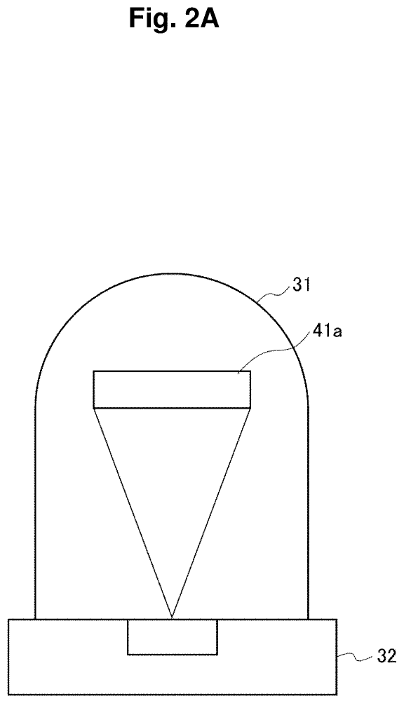

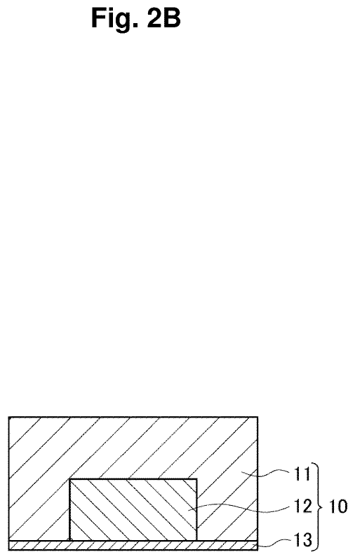

FIG. 1A is a cross-sectional view of a concrete block in which the metal is embedded, FIG. 1B is an explanatory drawing illustrating a cut surface L1 when the concrete block is cut to provide a concrete test piece, and FIG. 1C is a cross-sectional view of a concrete piece obtained by cutting the concrete block along the cut surface L1. FIG. 2A is an explanatory drawing illustrating a process of forming the metal film on the cut surface of the concrete piece, and FIG. 2B is an explanatory drawing illustrating a concrete test piece provided by forming the metal film.

Hereinafter, with reference to FIGS. 1A, 1B, 1C, 2A, and 2B, a procedure of a method for producing the concrete test piece according to an embodiment will be described.

First, as illustrated in FIG. 1A, a concrete block 41 in which a metal 12 is embedded in a concrete 11 is prepared. The metal 12 is, for example, iron (Fe). Although FIG. 1A illustrates an example of using the concrete block 41 in which the entire periphery of the metal 12 is covered with the concrete 11, at least a part of the metal 12 of the concrete block 41 should be covered with the concrete 11. Although the metal 12 and the concrete 11 have a rectangular parallelepiped shape in FIG. 1A, they may have a shape other than the rectangular parallelepiped shape.

Next, as illustrated in FIG. 1B, the planar cut surface L1 including the metal 12 is specified on the concrete block 41, and the concrete block 41 is cut along the cut surface L1. As a result, as illustrated in FIG. 1C, a concrete piece 41a provided by dividing the concrete block 41 into two pieces is produced. That is, the concrete piece 41a has the shape in which the metal 12 is exposed at the center and the periphery of the metal 12 is covered with the concrete 11.

As illustrated in FIG. 2A, the concrete piece 41a illustrated in FIG. 1C is inserted into a vacuum container 31, and the metal film is formed on the surfaces of the metal 12 and the concrete 11 using a vacuum evaporation method. For example, nickel (Ni) or palladium (Pd) may be used as the metal film.

Specifically, the metal film is formed on the surfaces of the metal 12 and the concrete 11 by disposing the metal such as the nickel (Ni) or the palladium (Pd) in an evaporation source 32, heating the metal, and evaporating the metal in the vacuum container 31. The method for forming the metal film is not limited to the vacuum evaporation method, and other film forming methods such as a sputtering method and a CVD method may be used.

As a result, as illustrated in FIG. 2B, the metal 12 is embedded in the surface of the concrete 11, and the concrete test piece 10 (Hereinafter, abbreviated as “test piece 10”) in which a metal film 13 is formed on the surfaces of the concrete 11 and the metal 12 is produced. Hereinafter, a surface of the test piece 10 on which the metal film 13 is formed (a lower surface of the test piece 10 illustrated in FIG. 2B) is referred to as a first surface, and a surface opposite to the first surface (an upper surface of the test piece 10 illustrated in FIG. 2B) is referred to as a second surface.

That is, the concrete test piece 10 according to the present embodiment includes the concrete 11 having the planar first surface, the metal 12 embedded in the first surface, and the metal film 13 formed on the first surface including the metal 12.

In addition, the concrete test piece 10 according to the present embodiment is produced by a step of cutting the concrete block 41 in which the metal 12 is embedded in the concrete 11 along the plane including the metal 12 to provide the concrete piece 41a, and the step of forming the metal film 13 on the metal 12 on the cut surface of the concrete piece 41a and the concrete 11.

In the present embodiment, the amount of hydrogen penetrating the metal 12 is measured using the test piece 10 produced by the above procedure. Hereinafter, a method for measuring the hydrogen content will be described.

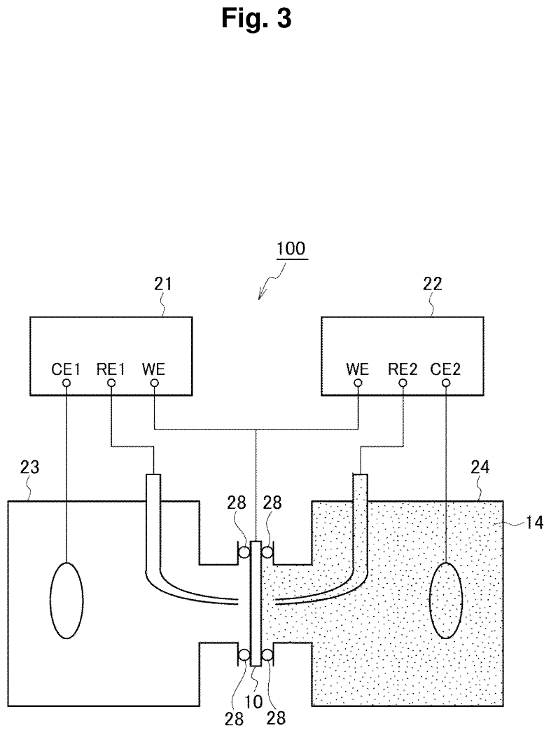

FIG. 3 is an explanatory drawing schematically showing a measurement device used for measuring the hydrogen content. As illustrated in FIG. 3, a measurement device 100 includes a first cell 23, a second cell 24, a first current detector 21, a second current detector 22, and fixtures 28.

The inside of the first cell 23 is an environment into which hydrogen penetrates. The inside of the first cell 23 is, for example, an outdoor environment. The outdoor environment is an outside air environment having an arbitrary temperature and an arbitrary humidity.

The second cell 24 is filled with a sodium hydroxide solution 14.

The test piece 10 is disposed in a connecting portion between the first cell 23 and the second cell 24, and is sealed and fixed by the fixtures 28. The test piece 10 is disposed such that the first surface is exposed to the second cell 24 and the second surface is exposed to the first cell 23. That is, the first surface of the test piece 10 is immersed in the sodium hydroxide solution 14.

The first current detector 21 is connected to a reference electrode RE1 and a counter electrode CE1 disposed in the first cell 23, and a sample electrode WE connecting to the test piece 10. The first current detector 21 measures a current flowing in the first cell based on a voltage generated in each of the electrodes RE1, CE1, and WE.

The second current detector 22 is connected to a reference electrode RE2 and a counter electrode CE2 disposed in the second cell 24, and the sample electrode WE connecting to the test piece 10. The second current detector 22 measures the current flowing through the sodium hydroxide solution 14 filled in the second cell based on the voltage generated in each of the electrodes RE2, CE2, and WE.

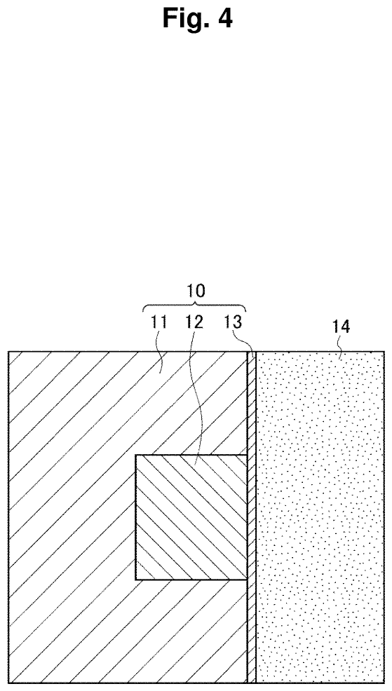

FIG. 4 is an explanatory drawing illustrating a state in which the first surface of the test piece 10 is in contact with the sodium hydroxide solution 14.

As illustrated in FIG. 4, the metal film 13 is formed on the surfaces of the metal 12 and the concrete 11 constituting the test piece 10. That is, the metal film 13 such as the nickel film or the palladium film is formed on the first surface of the test piece 10. The sodium hydroxide solution 14 does not permeate the nickel film or the palladium film. Therefore, the sodium hydroxide solution 14 filled in the second cell 24 does not permeate the concrete 11. As a result, it is possible to prevent the sodium hydroxide solution 14 from leaking into the first cell 23.

Next, the principle of measuring the hydrogen content by the measurement device 100 will be described. As illustrated in FIG. 3, the second surface (the surface on the left side in FIG. 4) of the test piece 10 is exposed to the outdoor environment in the first cell 23.

A part of the hydrogen existing in the first cell 23 penetrates the concrete 11 through the second surface of the test piece 10 and reaches the surface of the metal 12 (the surface on the left side in FIG. 4). Furthermore, a part of the hydrogen that has reached the surface of the metal 12 penetrates the metal 12, diffuses, and reaches the first surface (the right surface in FIG. 4) of the metal 12.

Hydrogen that has reached the first surface is ionized in the sodium hydroxide solution 14, and electrons are emitted by an ionization reaction. The second current detector 22 measures the current flowing in the sodium hydroxide solution 14.

The amount of hydrogen penetrating the metal 12 of the test piece 10 can be calculated based on the current measured by the second current detector 22.

In this way, the amount of hydrogen penetrating the metal (reinforcing bar) embedded and disposed in the concrete can be measured using the concrete test piece 10 simulating a structure of reinforced concrete.

As described above, the concrete test piece 10 according to the present embodiment includes the concrete 11 having the planar first surface, the metal 12 embedded in the first surface, and the metal film 13 formed on the first surface including the metal 12.

Since the metal film 13 is formed on the first surface which is the surface on the hydrogen detection side in the concrete test piece 10 according to the present embodiment, it is possible to prevent the sodium hydroxide solution 14 filled in the second cell 24 from permeating the concrete 11 and leaking to the first cell 23 when the hydrogen content is measured using the measurement device 100.

Therefore, it is possible to easily measure the amount of hydrogen in the metal embedded in concrete such as a reinforced concrete structure. According to the present embodiment, it is possible to non-invasively measure the corrosion state of the reinforcing bar embedded in the concrete.

In addition, since the nickel (Ni) or the palladium (Pd) is used as the metal film to be formed on the surface of the concrete 11 in the concrete test piece 10 according to the present embodiment, the leakage of the sodium hydroxide solution 14 can be more reliably prevented. Furthermore, the detection sensitivity of hydrogen can be improved by forming the film of nickel or palladium as the metal film.

In the present embodiment, the vacuum evaporation method or the like is adopted as the method for forming the metal film 13 on the surfaces of the concrete 11 and the metal 12. Therefore, since a plating treatment is not adopted as the method for forming the metal film of nickel, palladium, or the like, the problem that the plating solution permeates into the concrete can be avoided.

The present invention is not limited to the above embodiment, and various modifications can be made within the scope of the spirit of the present invention.

### REFERENCE SIGNS LIST

10 concrete test piece
11 concrete
12 metal
13 metal film
14 sodium hydroxide solution
21 first current detector
22 second current detector
23 first cell
24 second cell
28 fixture
31 vacuum container
32 evaporation source
41 concrete block
41a concrete piece
100 measurement device

1. A concrete test piece comprising:
a concrete having a planar first surface;
a metal embedded in the first surface; and
a metal film formed on the first surface including the metal.

2. The concrete test piece according to claim 1, wherein the metal film includes nickel or palladium.

3. A method for producing a concrete test piece, the method comprising the steps of:
cutting a concrete block in which a metal is embedded in a concrete on a plane including the metal to provide a concrete piece; and
forming a metal film on the metal and the concrete in a cut surface on the concrete piece.

4. The method for producing a concrete test piece according to claim 3, wherein the metal film is formed by a vacuum evaporation method.

5. The method for producing a concrete test piece according to claim 3, wherein the metal film includes nickel or palladium.

6. A method for measuring a hydrogen content, the method measuring an amount of hydrogen penetrating the metal in the concrete test piece according to claim 1, the method comprising the steps of:
immersing the first surface of the concrete in a sodium hydroxide solution and placing a second surface opposite the first surface in an outdoor environment to detect current flowing in the sodium hydroxide solution; and
calculating the amount of the hydrogen penetrating the metal based on the detected current.

7. The method for producing a concrete test piece according to claim 4, wherein the metal film includes nickel or palladium.

8. A method for measuring a hydrogen content, the method measuring an amount of hydrogen penetrating the metal in the concrete test piece according to claim 2, the method comprising the steps of:
immersing the first surface of the concrete in a sodium hydroxide solution and placing a second surface opposite the first surface in an outdoor environment to detect current flowing in the sodium hydroxide solution; and
calculating the amount of the hydrogen penetrating the metal based on the detected current.
